(19) United States
(12) Patent Application Publication
(10) Pub. No.: US 2026/0048213 A1
(43) Pub. Date: Feb. 19, 2026

(54) NASAL AND ORAL INHALERS

(71) Applicant: Flash Sales Inc., Miami, FL (US)

(72) Inventor: Man-Ning Hsieh, Taipei City (TW)

(21) Appl. No.: 19/217,632

(22) Filed: May 23, 2025

Related U.S. Application Data
(63) Continuation-in-part of application No. 18/807,332, filed on Aug. 16, 2024.

(51) Int. Cl. A61M 15/08; A61M 21/00; A61M 21/02

(52) U.S. Cl. CPC A61M 15/08; A61M 21/02; A61M 2021/0016

(57) ABSTRACT

Nasal and oral inhalers are disclosed herein. An example inhaler includes a body including a first end, a second end opposite the first end, and first and second nose tips on the first end. The first and second nose tips have first and second openings, respectively. The body also includes a third opening in the second end, and an internal cavity extending between the first and second openings and the third opening. The inhaler also includes a substance (e.g., an essential oil) in the internal cavity. The first and second nose tips are to be used to inhale vapors from the substance nasally, and the third opening is to be used to inhale vapors from the substance orally.

### RELATED APPLICATION

This patent arises from a continuation-in-part (CIP) of U.S. Application Ser. No. 18/807,332, titled “Nasal and Oral Inhalers,” filed Aug. 16, 2024. Priority is claimed to U.S. Application Ser. No. 18/807,332. U.S. Application Ser. No. 18/807,332 is hereby incorporated by reference in its entirety.

### FIELD OF THE DISCLOSURE

This disclosure relates generally to inhalers and, more particularly, to nasal and oral inhalers.

### BACKGROUND

Nasal inhalers are devices that contain essential oils or other substances that emit vapors that can be inhaled (breathed) into the user's nose. These nasal inhalers can be used for aromatherapy purposes. In particular, breathing in the vapors of the essential oil promotes different feelings or moods in the user, such as calming or energizing moods.

### BRIEF DESCRIPTION OF THE DRAWINGS

FIG. 1 is a perspective view of a first end of an example inhaler.

FIG. 2 is a perspective view of a second end of the example inhaler of FIG. 1.

FIG. 3 is a plan view of the first end of the example inhaler of FIG. 1.

FIG. 4 is a first side view of the example inhaler of FIG. 1.

FIG. 5 is a second side view of the example inhaler of FIG. 1.

FIG. 6 is a plan view of the second end of the example inhaler of FIG. 1.

FIG. 7 is a cross-sectional view of the example inhaler of FIG. 1 showing an example in which the inhaler includes two example pads with essential oil.

FIG. 8 is a cross-sectional view of the example inhaler of FIG. 1 showing an example in which the inhaler includes one example pad with essential oil.

FIG. 9 is a cross-sectional view of the example inhaler of FIG. 1 showing an example in which the inhaler includes an example automatic one-way check valve.

FIG. 10 is a cross-sectional view of the example inhaler of FIG. 1 showing an example in which the inhaler includes an example manually operable valve.

FIG. 11A is a perspective view of the example inhaler of FIG. 1 in which the inhaler includes an example mouthpiece.

FIG. 11B is a side view of the example inhaler of FIG. 11A.

FIG. 11C shows example caps on the example inhaler of FIGS. 11A and 11B.

FIG. 11D is a cross-sectional view of the example mouthpiece of FIG. 11A.

FIG. 12 is a perspective view of the first end of the example inhaler of FIG. 1 having a body with different side surfaces than the example in FIGS. 1-6.

FIG. 13 is a perspective view of the second end of the example inhaler of FIG. 12.

FIG. 14 is a plan view of the first end of the example inhaler of FIG. 12.

FIG. 15 is a first side view of the example inhaler of FIG. 12.

FIG. 16 is a second side view of the example inhaler of FIG. 12.

FIG. 17 is a third side view of the example inhaler of FIG. 12.

FIG. 18 is a fourth side view of the example inhaler of FIG. 12.

FIG. 19 is a plan view of the second end of the example inhaler of FIG. 1.

FIG. 20 is a perspective view of a first end of another example inhaler.

FIG. 21 is a perspective view of a second end of the example inhaler of FIG. 20.

FIG. 22 is a plan view of the first end of the example inhaler of FIG. 20.

FIG. 23 is a first side view of the example inhaler of FIG. 20.

FIG. 24 is a second side view of the example inhaler of FIG. 20.

FIG. 25 is a third side view of the example inhaler of FIG. 20.

FIG. 26 is a fourth side view of the example inhaler of FIG. 20.

FIG. 27 is a plan view of the second end of the example inhaler of FIG. 20.

FIG. 28 is a perspective view of a first end of another example inhaler.

FIG. 29 is a perspective view of a second end of the example inhaler of FIG. 28.

FIG. 30 is a side view of an example inhaler having an example roller.

FIG. 31 is a cross-sectional view of the example inhaler of FIG. 30.

FIG. 32 is an exploded view of the example roller of FIG. 30.

FIG. 33 shows example caps on the example inhaler of FIG. 30.

FIG. 34 is a cross-sectional view of a first example cap for the example inhalers disclosed herein.

FIG. 35A is a cross-sectional view of a second example cap for use with example inhalers disclosed herein having a mouthpiece.

FIG. 35B is a cross-sectional view of a second example cap for use with example inhalers disclosed herein having a roller.

In general, the same reference numbers will be used throughout the drawing(s) and accompanying written description to refer to the same or like parts. The figures are not necessarily to scale. Instead, the thickness of the layers or regions may be enlarged in the drawings. Although the figures show layers and regions with clean lines and boundaries, some or all of these lines and/or boundaries may be idealized. In reality, the boundaries and/or lines may be unobservable, blended, and/or irregular.

### DETAILED DESCRIPTION

Disclosed herein are example inhalers that can be used to inhale vapors from a substance nasally or orally. In particular, the example inhalers disclosed herein includes one or more nose openings to enable a user to sniff and/or otherwise breathe in the vapors through their nose and one or more mouth openings that enable a user to suck and/or otherwise breathe in the vapors through their mouth. This enables a user to be able to enjoy the effects of the vapors nasally or orally.

The example inhalers disclosed herein can be used for aromatherapy, for example. Aromatherapy is the practice of using essential oils and/or other compounds for therapeutic benefit. Essential oils, also known as volatile oils, ethereal oils, or aetherolea oils, may be a natural oil or concentrated hydrophobic liquid having a characteristic fragrance of a plant or other source from which it is extracted. Essential oils may also contain additives, preservatives, and/or other synthetics to produce a certain fragrance. Aromatherapy is a natural way to promote relaxation, improve mode, and enhance overall well-being with the use of aromas from such oils. The inhaler can be filled with different essential oils for different uses. In particular, different essential oils may promote different feelings or moods in the user.

For example, lavender scents have been shown to promote better sleep, peppermint scents have been shown to boost energy, lemon scents have been shown to reduce anxiety, and rosemary scents have been shown to improve focus and concentration. Further, the example inhalers disclosed herein can be used for medicinal purposes. For example, the inhalers can contain substances that emit vapors that help to relieve congestion and/or reduce allergies. The inhalers can also be used to dispense nicotine. Therefore, while many of the examples disclosed here are described in connection with essential oils, it is to be understood that the liquid or substance in the inhaler can be any other type of liquid or substance.

As mentioned above, the example inhalers disclosed herein have one or more openings that enable a user to inhale the vapors nasally or orally. Therefore, the example inhalers give the user the option on how to enjoy the essential oil, nasally or orally. The mouth option may be used to help users that traditionally vape or smoke to ease or substitute their cravings. In other words, the mouth options act as a substitute for the oral fixation of vaping or smoking, thereby helping the user to quit or transition away from vaping or smoking.

An example inhaler disclosed herein includes a body that has a first end with two nose tips and a second end, opposite the first end, with a mouth opening. The body has an internal cavity with a substance that emits vapors or fumes that can be inhaled. The substance is typically a liquid, such as an oil (e.g., an essential oil) or gel (e.g., petroleum jelly), but can also be a solid (e.g., a block of cedar wood, coffee beans, salts). The internal cavity is connected to or in fluid communication with the nose tips and the mouth opening. Therefore, the same essential oil can be enjoyed nasally, via the nose tips, or orally, via the mouth opening.

Also disclosed herein are example inhalers that include a body that has a first end with two nose tips and a second end with a roller. The nose tips can be used to nasally inhale the vapors or fumes from a substance (e.g., an essential oil) in an internal cavity of the inhaler, while the roller can be used to dispense a liquid. For example, the roller can be used to apply a liquid such as an essential oil to the skin of the user or another surface (e.g., clothing). Applying certain liquids, such as essential oils, to the skin has been shown to have many benefits, such as reducing inflammation, reducing itchiness, reducing dryness, and/or reducing wrinkles, to name a few. In another example, the roller can be used to dispense or apply the liquid to a user's clothing to provide a fragrance or scent to the clothing. In some examples, the roller can be removed from the second end of the inhaler and the second end can be used to orally inhale the fumes or vapors from the substance in the inhaler. Therefore, the example inhaler provides the user with multiple options for experiencing the substance.

In some examples disclosed herein the body of the inhaler is V-shaped. For example, the body has a first portion and a second portion. The first and second portions are coupled or merged at the second end (where the mouth opening is located) and are angled away from each other toward the first end (where the nose tips are located). The V-shaped body results in a smaller, more compact design that is more portable and easier for a user to carry in their pockets or bags (e.g., purses, backpacks, etc.). The V-shaped body also improves air flow to the nose tips and the mouth opening, thereby enhancing the aromatherapy experience.

The example inhalers disclosed herein do not require a battery or any heating element. Instead, the fumes or vapors from the substance are merely inhaled naturally by sniffing the fumes through the user's nose or breathing in the fumes through the user's mouth. Therefore, the inhalers are relatively light and inexpensive. As used herein, the terms vapor(s) and fume(s) are defined as the particles or molecules of a substance (e.g., a solid such as wood, a liquid such as an essential oil) that are in a gaseous state and suspended in the air. The terms vapor(s) and fume(s) do not require heating or temperature change of the underlying substance.

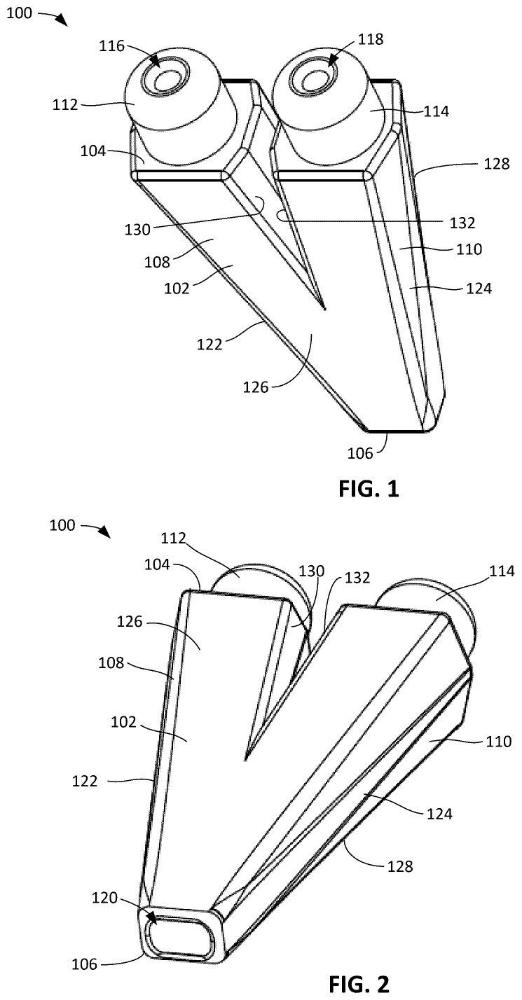

FIGS. 1 and 2 are perspective views of opposite ends of an example inhaler 100 constructed accordance with the teachings of this disclosure. The example inhaler 100 can be used to inhale vapors from a substance (e.g., an essential oil) nasally or orally, as disclosed in further detail herein.

The inhaler 100 includes an example body 102. The body 102 has a first end 104 and a second end 106 opposite the first end. The first and second ends 104, 106 may be referred to as top and bottom ends depending on the mode of use and/or orientation of the inhaler 100. In the illustrated example, the body 102 is V-shaped. In particular, the body 102 includes a first portion 108 and a second portion 110, which may also be referred to branches or stems. The first and second portion 108, 110 are coupled and/or merged at the second end 106. The second end 106 forms a vertex. The first and second portions 108, 110 angle away from each other toward the first end 104.

The body 102 includes a first example nose tip 112 and a second example nose tip 114 on the first end 104. The first nose tip 112 extends outward from the first portion 108 and the second nose tip 114 extends outward from the second portion 110. The first and second nose tips 112, 114 can be coupled to or integrally formed with the first and second portions 108, 110. In the illustrated example, the first and second nose tips 112, 114 are cylindrical shaped, which enables the nose tips 112, 114 to be comfortably inserted into a user's nostrils, but in other examples can be shaped differently. The first and second nose tips 112, 114 are dimensions to fit within the nostrils of an adult human. In other examples, the first and second nose tips 112, 114 can have different dimensions and/or be coupled to reduction adapters for use with children. The first and second nose tips 112, 114 have first and second openings 116, 118, respectively, that connect to and/or otherwise in fluid communication with an internal cavity in the body 102 that contains a substance. To use the inhaler nasally, a user can hold the body 102 (e.g., with their hand) and move the inhaler 100 toward their nose such that the nose tips 112, 114 are inserted into their nostrils or located relatively close to their nostrils. Then, when the user breathes in or inhales, the fumes or vapors from the substance are breathed from the openings 116, 118 and into the user's nose. Breathing in the vapors or fume can promote various moods or feelings in the user.

The body 102 can be constructed of plastic (e.g., acrylonitrile butadiene styrene (ABS)), metal, or another material. In some examples, the body 102 is constructed as a single unitary part or component, such as a monolithic structure. For example, the body 102 may be molded as a single piece of plastic. In other examples, the body 102 can be constructed of separate parts or components that are coupled together, for example, via fasteners, glue, welding, etc. In some examples, the body 102 can be constructed via additive manufacturing (e.g., three-dimensionally printed). In some examples, the V-shaped portion of the body 102 is constructed of a softer plastic, such as acrylonitrile butadiene styrene (ABS), and the nose tips 112, 114 are constructed of a harder plastic, such as polypropylene (PP).

The example inhaler 100 can also be used as an oral inhaler. As shown in FIG. 2, the body 102 has a third opening 120, referred to herein as a mouth opening 120, in the second end 106 of the body 102. The mouth opening 120 is also connected to and/or otherwise in fluid communication with the internal cavity. To use as an oral inhaler, a user can insert the second end 106 of the body 102 into their mouth and inhale. The fumes or vapors from the substance (e.g., essential oil) are aspirated into the user's mouth and/or lungs. Therefore, the example inhaler 100 can be used to ingest or experience the substance via nose or mouth. The example inhaler 100 does not include or require any batteries or electronics. Thus, the example inhaler is relatively light and simple to use.

While the mouth opening 120 is formed in the second end 106, opposite the first end 104, in other examples, the mouth opening 120 can be formed in another side surface of the body 102 that is not opposite the first end 104.

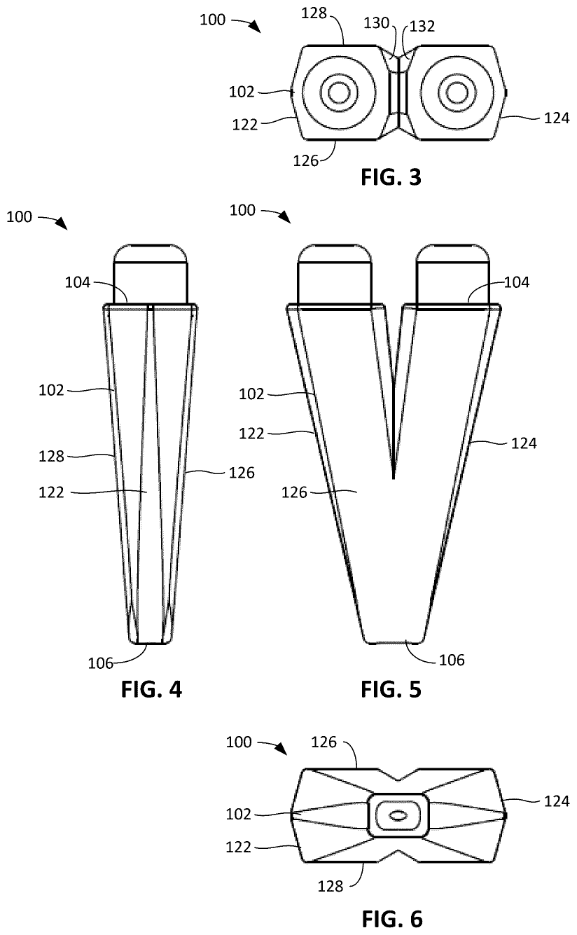

FIGS. 3-6 are different views of the example inhaler 100. The inhaler 100 is symmetrical about a vertical plane. As such, the second outer side surface 124 appears the same as the first outer side surface 122 in FIG. 4, and the fourth outer side surface 128 appears the same as the third outer side surface 126 of FIG. 5. As mentioned above, the body 102 of the inhaler 100 is V-shaped. As such, the shape is designed to converge or narrow from the nose tips 112, 114 on the first end 104 toward the mouth opening 120 on the second end 106.

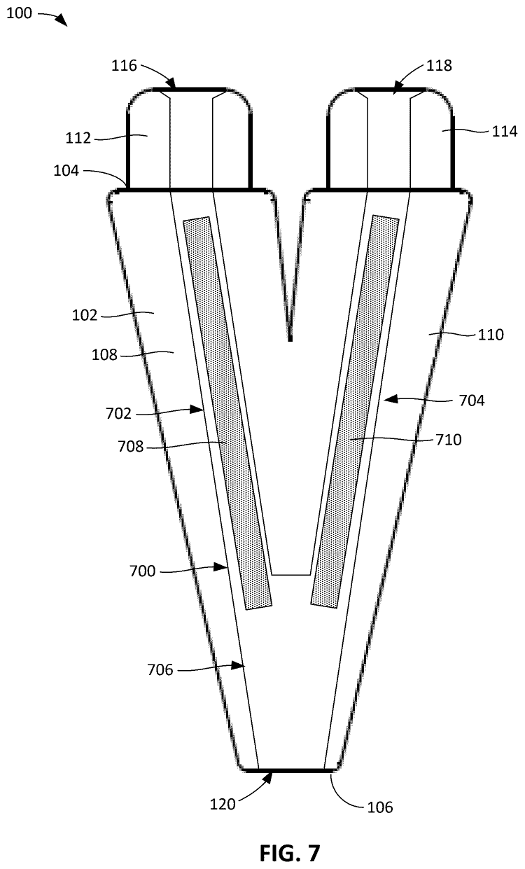

FIG. 7 is a cross-sectional view of the inhaler 100 of FIGS. 1-6. As shown in FIG. 7, the body 102 defines or forms an internal cavity 700.

The internal cavity 700 is fluidly coupled to and extends between the first and second openings 116, 118 of the nose tips 112, 114 on the first end 104 and to the mouth opening 120 on the second end 106. The internal cavity 700 may also be referred to as a channel, chamber, or compartment. In the illustrated example, the internal cavity 700 has one or more portions or sections. For example, the internal cavity 700 has a first channel 702 that extends through the first portion 108 of the body 102 to the first opening 116 of the first nose tip 112, a second channel 704 that extends through the second portion 110 of the body 102 to the second opening 118 of the second nose tip 114, and a common chamber portion 706 that is fluidly coupled to the first and second channels 702, 704 and extends to the mouth opening 120.

The inhaler 100 includes a substance in the internal cavity 700 that emits vapors or fumes that are be inhaled by the user. In some examples, the substance is a liquid, such as an essential oil or other aromatherapy oil. The essential oil may have a particular fragrance for invoking certain moods or feelings in the user. In some examples, the inhaler 100 includes an absorbent material (e.g., a pad or substrate) that is soaked in the liquid. For example, the absorbent material may be constructed of a plant-based fiber such as, for example, rattan, bamboo, and/or cotton (e.g., a cotton swab). In some examples, the absorbent material may be a foam material made of polypropylene or polyurethane, a paper substrate, a polyester rod, and/or a combination of materials. For example, in the illustrated example, the inhaler 10 includes two example substrates or pads, including a first pad 708 and a second pad 710. The first pad 708 is at least partially disposed in the first channel 702 of the internal cavity 700 and the second pad 710 is at least partially disposed in the second channel 704 of the internal cavity 700. The first and second pad 708, 710 are constructed of the absorbent material (e.g., cotton) and are soaked or saturated in a liquid (e.g., an essential oil). Gaseous vapors or fumes are emitted from the first and second pads 708, 710. The vapors or fumes can be inhaled nasally, using the nose tips 112, 114, or orally, using the mouth opening 120. In some examples, using two separate pads in the two channels 702, 704 provides a stronger (e.g., maximum) experience for the user. In some examples, the pads 708, 710 are implemented as polyester rods that are soaked in the liquid (e.g., an essential oil).

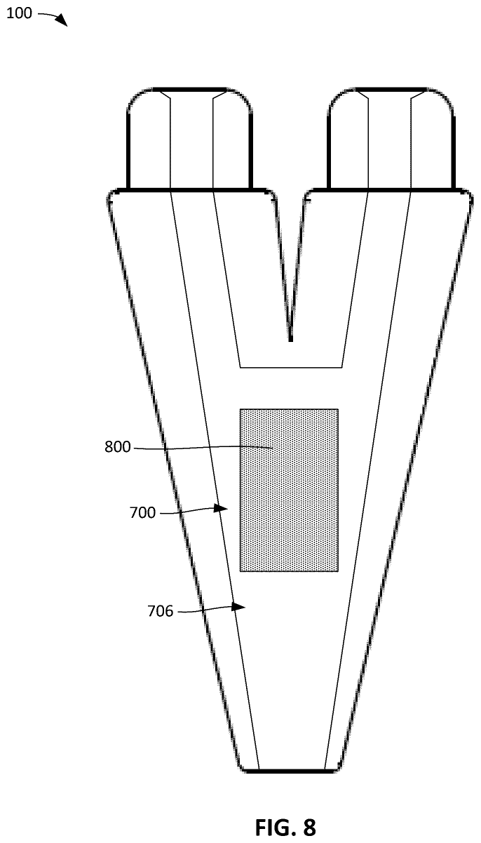

In other examples, the absorbent material with essential oil can be disposed in another location. For example, FIG. 8 shows an example in which the inhaler 100 includes an example pad 800 in the common chamber portion 706 of the internal cavity 700. The pad 800 is constructed of an absorbent material that is soaked in the liquid substance (e.g., an essential oil). While only one pad 800 is shown, the inhaler 100 can include any number of separate substrates or pads, such as two, three, four, five, etc. In some examples, the pad(s) are held in the internal cavity 700 via friction fit. In other examples, the pad(s) can be coupled to the body 102 by various coupling techniques, such as using a mechanical fastener or a chemical fastener (e.g., an adhesive such as glue). In other examples, the liquid may not be soaked into a pad. Instead, the liquid may be a gel that can be disposed in a certain area or applied along the inner surfaces of the internal cavity 700.

In some examples, the liquid substance in the inhaler 100 may be effective for a certain period of time and/or for a certain number of uses (e.g., puffs or inhales). For example, an essential oil may be best used within 30 days of first opening for maximum strength of the vapors. Additionally or alternatively, the essential oil may be effective for about 100-200 uses. After that, the inhaler 100 can be discarded. In other examples, the pad(s) and/or liquid can be easily replaceable, such that a manufacturer or a user can remove and replace the pad(s) and/or liquid.

While in some examples the substance is a liquid (e.g., an essential oil), in other examples, the inhaler 100 can include a substance that is a solid material. For example, the substance in the inhaler 100 can be coffee beans, salts, minerals, herbs, spices, and/or other scented natural or synthetic materials

In some examples, a certain amount of airflow is to be used when using the inhaler 100 orally, but not when using the inhaler 100 nasally. In other words, in some examples, a first amount of airflow is used when operating the inhaler 100 orally, and a second amount of airflow (e.g., no airflow) that is different than the first amount of airflow is used when operating the inhaler 100 nasally. For example, it may be desired to allow some airflow through the internal cavity 700 when a user is inhaling the fumes orally to ensure enough vapor or fumes can be consumed and sensed in the user's mouth and/or lungs. However, when using nasally, additional airflow could dilute the vapors or fumes too much and therefore inhibit the intended purpose.

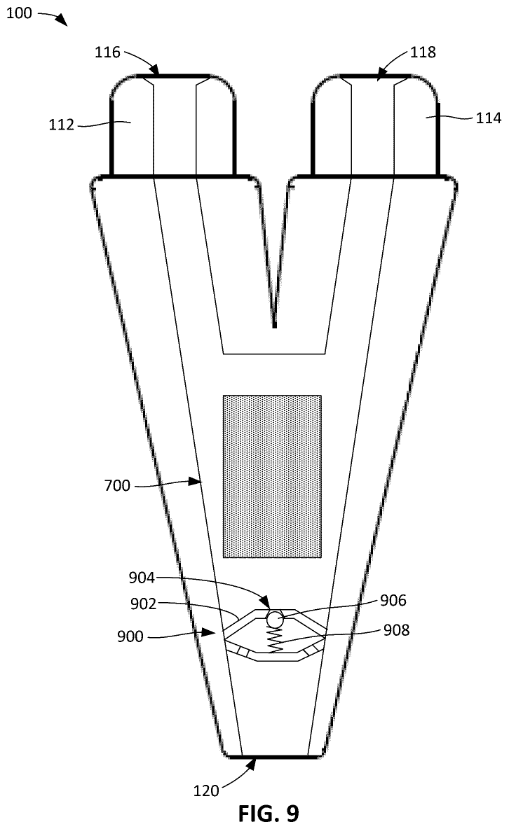

FIG. 9 illustrates an example in which the inhaler 100 includes an example valve 900 in the internal cavity 700. The valve 900 is operable between an open state and a closed state. Therefore, the valve 900 can operate between an open state to airflow in one direction and a closed state to block airflow in the opposite direction. For example, when a user is using the inhaler 100 orally and applying suction on the mouth opening 120, the valve 900 is opened. This allows outside air to be sucked through the openings 116, 118 of the nose tips 112, 114, and through the internal cavity 700 to the mouth opening 120. Conversely, when a user is using the inhaler 100 nasally and applying suction at the nose tips 112, 114, the valve 900 is closed, and therefore blocks airflow through the internal cavity 700.

In the illustrated example, the valve 900 is a one-way check valve, which is a valve that automatically allows airflow in one direction and blocks airflow in the opposite direction. The valve 900 is disposed in the internal cavity 700 between the liquid (which, in this example, is an essential oil soak into the pad 800) and the mouth opening 120. In some examples, the valve 900 is normally closed (e.g., biased to a closed state). The valve 900 automatically opens when a vacuum or suction is applied on the mouth opening 120. Therefore, when a user inhales from the mouth opening 120, the valve 900 automatically opens and allows airflow through the internal cavity 700. However, when the user stops inhaling on the mouth opening 120, the valve 900 automatically closes. As such, when a user applies suction via their nose on the nose tips 112, 114, the valve 900 remains closed and blocks airflow through the internal cavity 700. While the example valve 900 is shown in connection with the pad 800, the valve 900 can similarly be used in connection with an inhaler having multiple pads, such as the example shown in FIG. 7. In some examples, when using the inhaler 100 orally, the user can place one of their fingers over one of the nose tips 112, 114 to block one of the flow paths, which reduces or restricts the amount of airflow through the inhaler 100 to the user's mouth. This enables a user to at least partially control the amount of airflow when using the inhaler 100 orally.

In the illustrated example, the valve 900 includes a valve body 902 with a flow passage 904, and a ball 906 that is biased by a spring 908 into the flow passage 904. When a suction or vacuum is applied at the mouth opening 120, the ball 906 is pulled out of the flow passage 904 to enable airflow through the internal cavity 700. When no suction is applied at the mouth opening 120, and/or suction is applied at the nose tips 112, 114, the ball 906 is biased into and closes the flow passage 904, thereby blocking airflow through the internal cavity 700. In other examples, the valve 900 can be implemented by another type of one-way check valve, such as a shim plate valve or a reed valve.

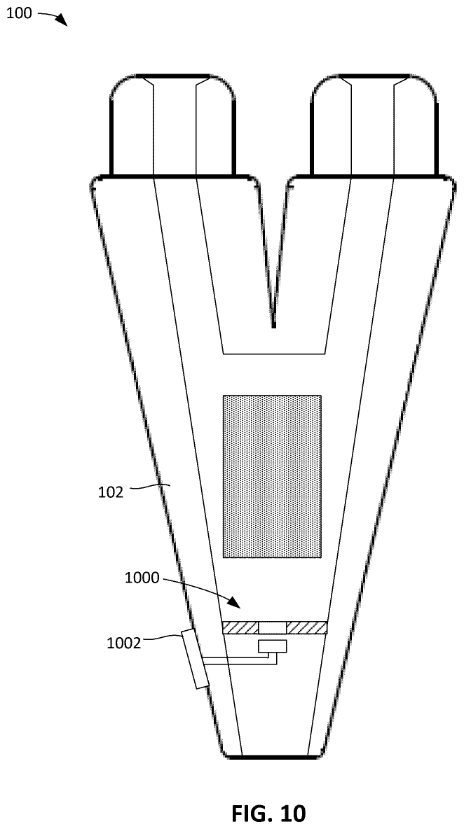

In some examples, the inhaler 100 may include a manually-operable valve. For example, FIG. 10 shows an example of the inhaler 100 including an example valve 1000 in the internal cavity 700 of the body 102.

In the illustrated example, the inhaler 100 includes an example switch or button 1002 that is operable to open or close the valve 1000. In some examples, the button 1002 is slidable along an outer surface of the body 102. A user may slide the button 1002 in one direction to open the valve 1000 and slide the button 1002 in the opposite direction to close the valve 1000. In other examples, the button 1002 actuated by pressing the button 1002 or moving the button 1002 in other directions. In some examples, the button 1002 is biased (e.g., via a spring) in the closed direction. Therefore, the user only needs to move the button 1002 to open the valve 1000, and then the button 1002 and the valve 1000 automatically move back to the closed state to control the airflow as discussed above in connection with FIG. 9.

In other examples, instead of including a valve, a user may use their finger to cover/block the mouth opening 120 when using the inhaler 100 nasally. This prevents air from flowing into the mouth opening 120 and through the internal cavity 700. In some examples, the inhaler 100 may include a cap or plug that can be used to seal the mouth opening 120 when the inhaler 100 is being used nasally, thereby preventing airflow through the mouth opening 120. In some examples, the inhaler 100 may include caps or plugs for the nose tips 112, 114. The caps can be used to seal the nose tips 112, 114 when the inhaler 100 is being used orally. Additionally, one cap can be used to block one of the nose tips 112, 114 if it is desired to use the inhaler 100 for one nasal passage (e.g., to deliver congestion relief to one nasal passage). The caps or plugs can also be used to protect the openings 116, 118, 120 from contaminants when the inhaler 100 is not being used.

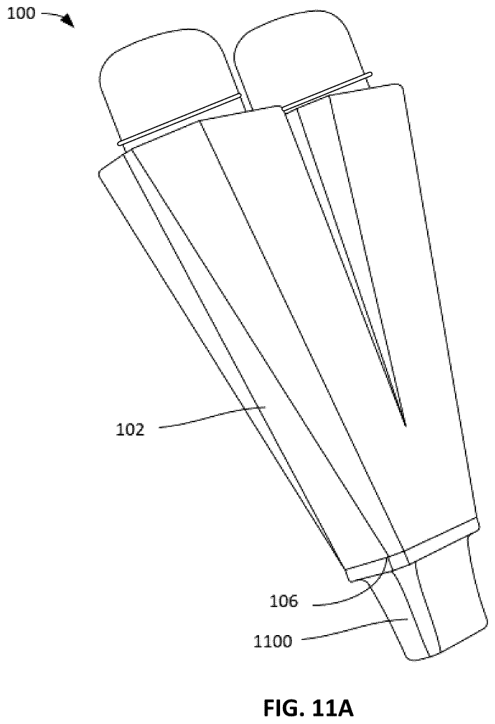

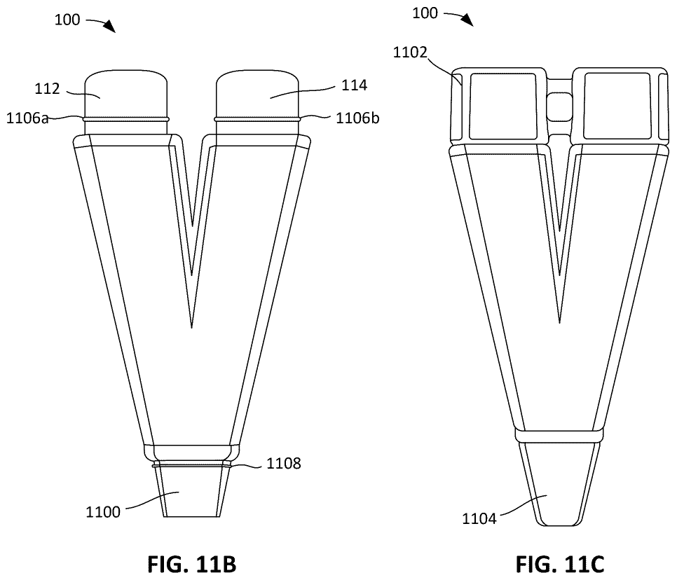

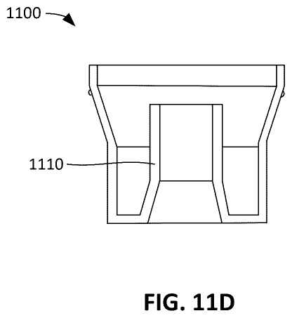

FIG. 11A shows an example in which the inhaler 100 includes an example mouthpiece 1100. The mouthpiece 1100 is coupled to and extends outward from the second end 106 of the body 102. In some examples, the mouthpiece 1100 is removably coupled to the second end 106, such that a user can attach the mouthpiece 1100 during use and remove the mouthpiece 1100 when not being used. For example, the mouthpiece 1100 can be coupled to the second end 106 via friction fit (e.g., a portion of the mouthpiece 1100 is friction fit into the mouth opening 120). The mouthpiece 1100 has a tapered and elongated shape, which is easier and more comfortable for a user to place and grasp between their lips. FIG. 11B is a side view of the example inhaler 100 of FIG. 11A with the example mouthpiece 1100.

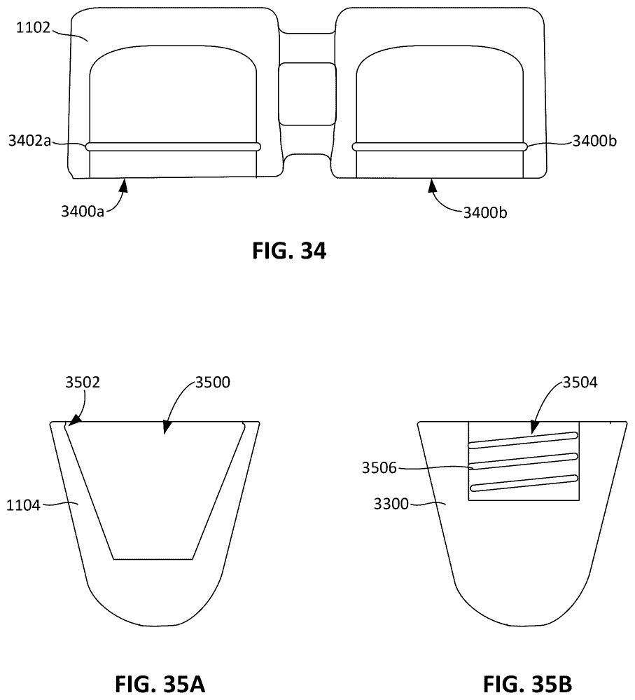

As disclosed above, in some examples, the inhaler 100 can include one or more caps. For example, FIG. 11C shows an example in which a first cap 1102 is placed on and covers the nose tips 112, 114 (FIG. 11B), and a second cap 1104 is placed on and covers the mouthpiece 1100 (FIG. 11B). The caps 1102, 1104 can be used when the user is not using the inhaler 100, such as when the user is transporting/carrying the inhaler 100 in the user's pocket or purse. The caps 1102, 1104 help to keep foreign debris (e.g., dust, dirt, etc.) out of the inhaler 100, as well as help prolong the life of the internal substance(s) (e.g., oil). In some examples, the caps 1102, 1104 are removably coupled to the inhaler 100 via friction fit and/or a snap fit. For example, as shown in FIG. 11B, the nose tips 112, 114 have rings 1106a, 1106b that protrude outward from the respective nose tips 112, 114. When the first cap 1102 is placed over the nose tips 112, 114, the rings 1106a, 1106b engage the inner surface of the first cap 1102, which holds the first cap 1102 on the nose tips 112, 114 until a sufficient force is used to remove the first cap 1102 (e.g., a user pulling on the first cap 1102). Therefore, the first cap 1102 can be removably coupled to the nose tips 112, 114. A cross-sectional view of the first cap 1102 is disclosed in further detail in connection with FIG. 34. Similarly, the mouthpiece 1100 includes a ring 1108 that protrudes outward from the mouthpiece 1100. When the second cap 1104 is placed on the mouthpiece 1100, the ring 1108 engages the inner surface of the second cap 1104 to hold the second cap 1104 on the mouthpiece 1100. Therefore, the second cap 1104 can be removably coupled to the mouthpiece 1100. A cross-sectional view of the second cap 1104 is disclosed in further detail in connection with FIG. 35A. In other examples, the inhaler 100 may not include the rings 1106a, 1106b, 1108. Instead, the caps 1102, 1104 may be sized to directly engage the sides of the nose tips 112, 114 and the mouthpiece 1100, respectively, to hold the caps 1102, 1104 in place. In some examples, the caps 1102, 1104 can be constructed of a flexible or stretchable material, such as silicone, that can be flexed to fit over the nose tips 112, 114 and the mouthpiece 1100, respectively. Additionally or alternatively, in other examples, other mechanisms can be used to hold the caps 1102, 1104 on the inhaler 100, such as a threaded connection, removable pins, latches, such snap-buttons, or hook and loop fasteners.

FIG. 11D is a cross-sectional view of the mouthpiece 1100. In some examples, the mouthpiece has an internal throat 1110 that increases air resistance, which enhances the user's experience when inhaling through the mouth.

While in the example of FIGS. 11A-11C the inhaler 100 includes a mouthpiece 1100, in other examples, the inhaler 100 may not include a mouthpiece. Instead, the user may place their lips directly on the body 102 at the around the mouth opening 120 (FIG. 2).

Referring back to FIGS. 1-6, the body 102 of the inhaler 100 is V-shaped and includes the first and second portions 108, 110. In the illustrated example, the first and second portions 108, 110 are polyhedrons (e.g., rectangular or cuboid shaped). The first and second portions 108, 110 extend from a rectangular prism at the second end 106 and diverge into separate polyhedrons at the first end 104. In other examples other shapes including other polyhedrons may be used. In the illustrated example, the body 102 has the first end 104, the second end 106 opposite the first end 104, a first outer side surface 122, a second outer side surface 124 oppose the first outer side surface 122, a third outer side surface 126, and a fourth outer side surface 128. The body 102 also has a first inner side surface 130 and a second inner side surface 132 that form a gap between the first and second portions 108, 110. In the illustrated example, the first outer side surface 122, the second outer side surface 124, the first inner side surface 130, and the second inner side surface 132 are faceted. In particular, the surfaces 122, 124, 130, 132 each have multiple distinct planes or surfaces. In some examples, the corners or edges between any of the ends 104, 106 and/or the surfaces 122, 124, 126, 128, 130, 132 are rounded or curved.

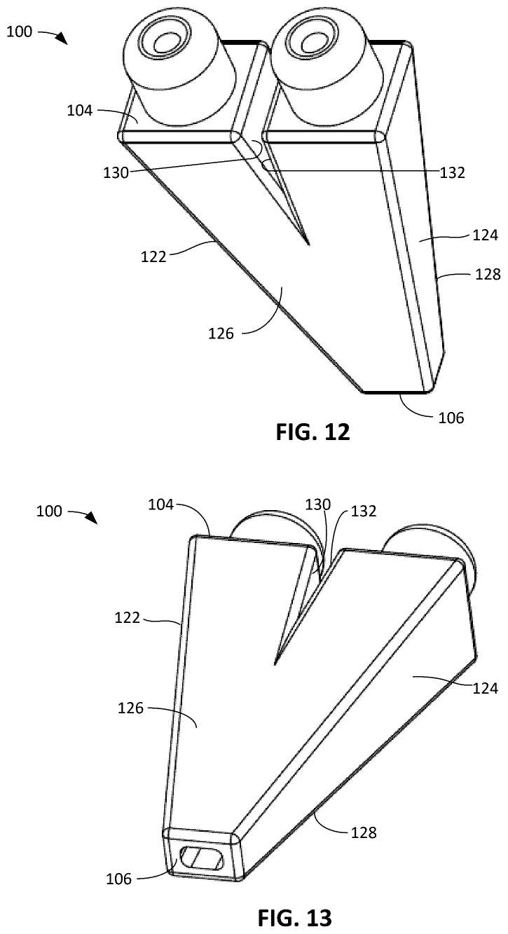

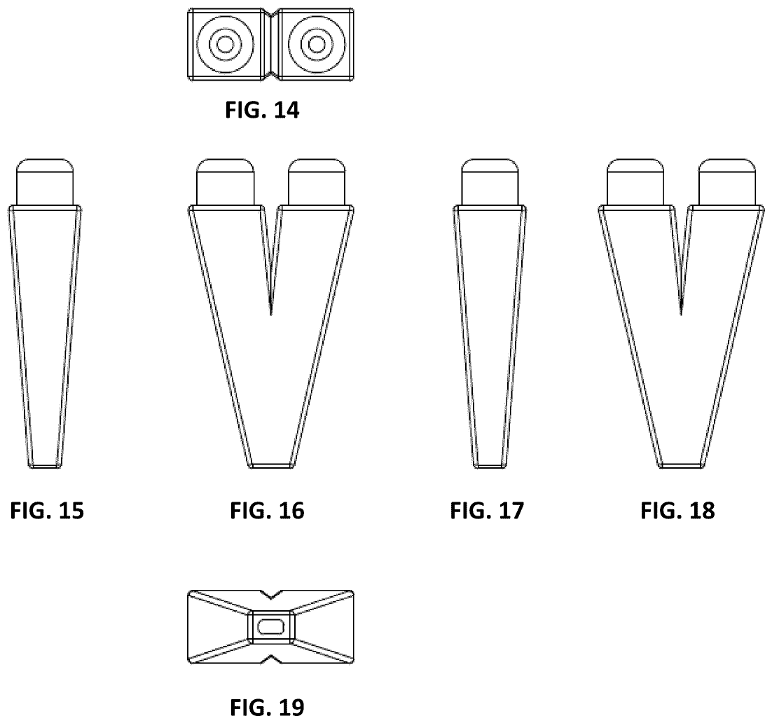

In other examples, the body 102 can be shaped differentially. For example, FIGS. 12 and 13 are perspective views showing an example in which the first outer side surface 122, the second outer side surface 124, the first inner side surface 130, and the second inner side surface 132 are not faceted. Instead, each of the surfaces 122, 124, 130, 132 is a single planar surface. In the example of FIGS. 12 and 13, the edges between the ends 104, 106 and the surfaces 122, 124, 126, 128, 130, 132 are rounded. FIGS. 14-19 show the six side views of the inhaler 100 of FIGS. 12 and 13.

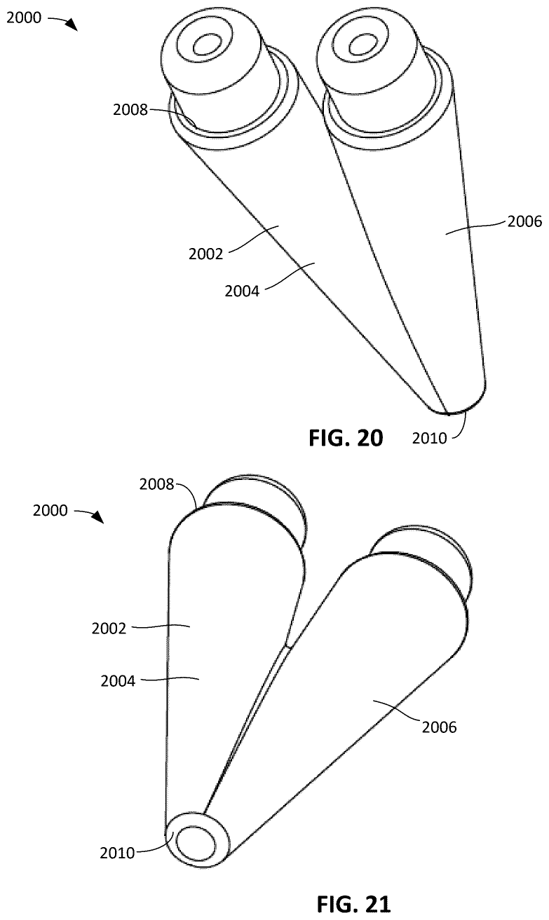

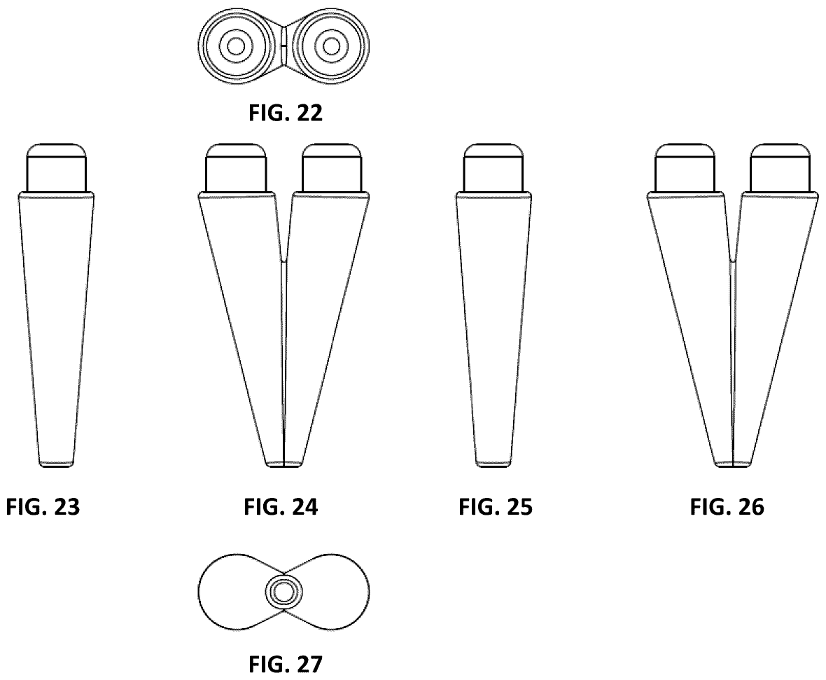

FIGS. 20 and 21 are perspective views another example inhaler 2000 with a V-shaped body 2002 having a first portion 2004 and a second portion 2006. Any of the example aspects disclosed above in connection with FIGS. 1-19 can likewise apply to the inhaler 2000. In the illustrated example of FIGS. 20 and 21, the first and second portions 2004, 2006 are conical. In particular, the first and second portions 2004, 2006 are cylinders that decrease in diameter from a first end 2008 to a second end 2010, where the first and second portions 2004, 2006 merge. This results in a small, streamlined profile that is lightweight and easily portable. FIGS. 22-27 show the six side views of the inhaler 2000 of FIGS. 20 and 21.

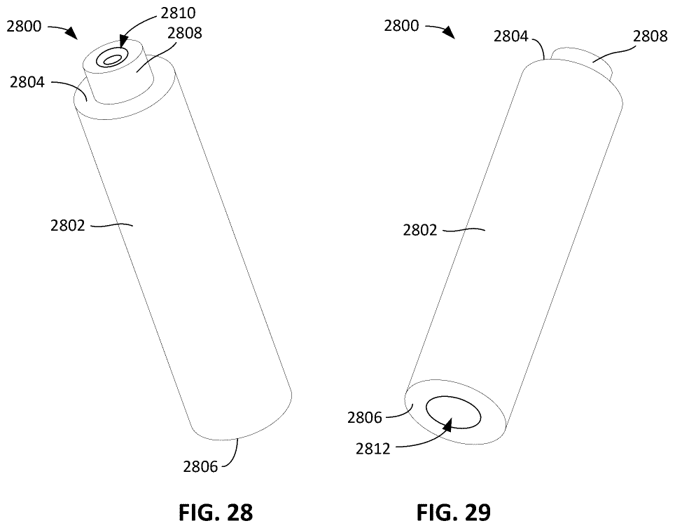

While in the examples disclosed above the inhalers include two nose tips, in other examples, an inhaler may include only one nose tip. For example, FIGS. 28 and 29 are perspective views of an example inhaler 2800 having a body 2802 with a first end and a second end 2806 opposite the first end 2804. In this example, the inhaler 2800 has one nose tip 2808 with an opening 2810 on the first end 2804 that is connected to the internal cavity. Therefore, in this example, the inhaler 2800 may be used to inhale the vapors through only one of the user's nostrils. The inhaler 2800 has a mouth opening 2812 in the second end 2806, which may be used to inhale the vapors orally. In the illustrated example, the body 2802 is relatively small and narrow. In this example, the body 2802 is cylindrical, but in other examples can be shaped differently. Any of the example aspects disclosed in connection with the other inhalers 100, 2000 of FIGS. 1-27 can likewise apply to the example inhaler 2800.

While the example inhaler 100 disclosed above has a mouth opening 120 for using the inhaler 100 orally, in other examples, the inhaler 100 may not include a mouth opening 120. Instead, the inhaler 100 may only include the nose tips 112, 114 for using the inhaler 100 nasally. In other words, the inhaler 100 may only be a nasal inhaler. The second end 106 may be closed or flat. The V-shaped body 102 enables two substances, such as two the two pads 708, 710, to be used for the separate channels to the nose tips 112, 114, which increases (e.g., maximizes) the experience for the user.

In some examples disclosed herein, an inhaler can include means for applying a liquid, such as an essential oil, to the skin of a user.

Applying an essential oil to the skin has many benefits, such as reducing inflammation, reducing itchiness, reducing dryness, and/or reducing wrinkles, to name a few.

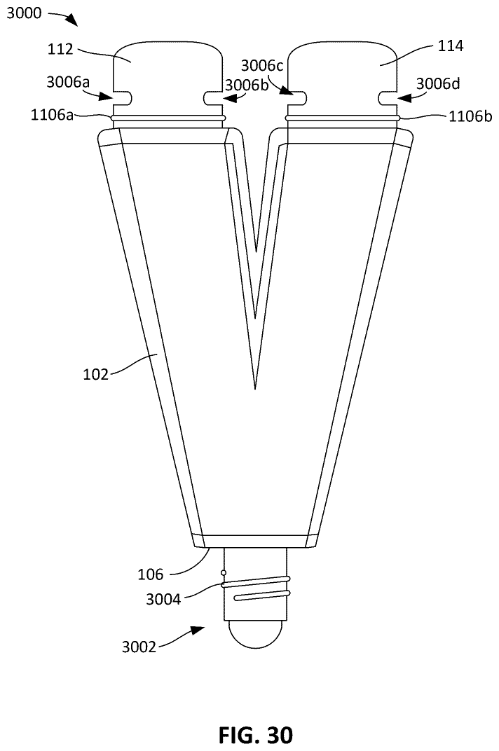

FIG. 30 illustrates an example inhaler 3000 that has the same or similar V-shaped body 102 with the nose tips 112, 114 as described in connection with FIGS. 1-19. However, instead of using the second end 106 as an oral inhaler, the inhaler 3000 includes a roller 3002 on the second end 106 of the body 102. The roller 3002 contains a liquid (e.g., an essential oil). The roller 3002 can be used to dispense (e.g., apply, roll, distribute, etc.) the liquid to skin of a user. Therefore, in this example the inhaler 3000 includes a nasal inhaler (the nose tips 112, 114) for nasally consuming the vapors or fumes of a substance, and includes a roller 3002 for applying a liquid to the skin of the user. The roller 3002 can also be used to dispense the liquid onto other types of items or surfaces, such as clothing. While the example roller 3002 is described in connection with the inhaler 3000, it is understood the example roller 3002 can be used on any of the example inhalers disclosed herein.

In the illustrated example of FIG. 30, the roller 3002 is coupled to and extends outward (e.g., downward) from the second end 106 of the body 102. In the illustrated example, the roller 3002 includes threads 3004 that can be used to attach a cap to the roller 3002, as disclosed in further detail herein. In the illustrated example, the first and second nose tips 112, 114 have air flow openings 3006a, 3006b, 3006c, 3006d (e.g., slots). The openings 3006a-3006d allow air flow into the internal cavity 700 (FIG. 7) through the nose tips 112, 114 when the inhaler 3000 is being used nasally, because the second end 106 is now blocked by the roller 3002. In this example, each nose tip 112, 114 includes two openings. In other examples, each nose tip 112, 114 may include only one opening or more than two openings. Further, while in the example of FIG. 30 the inhaler 3000 has two nose tips, in other examples, the inhaler 3000 may only have one nose tip.

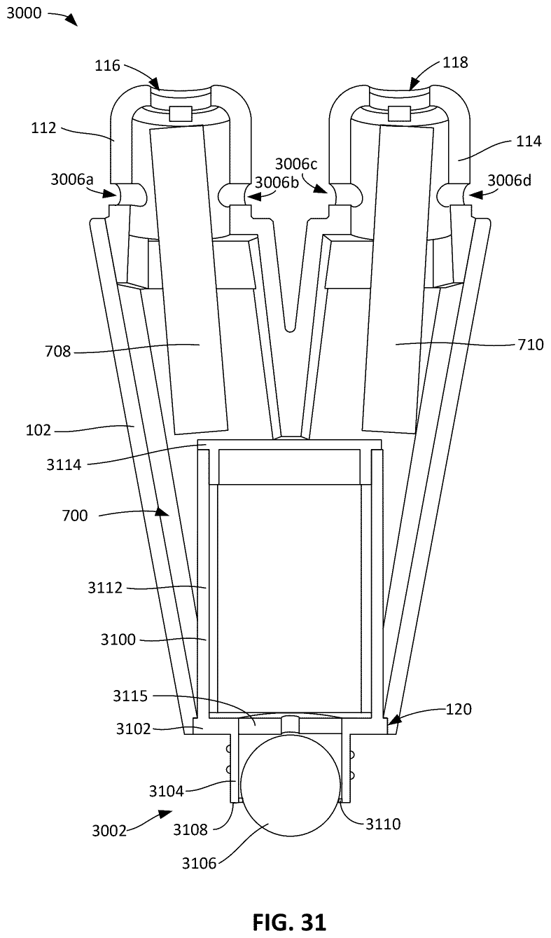

FIG. 31 is a cross-sectional view of the inhaler 3000. The roller 3002 includes a body 3100 coupled to the second end 106 of the body 102. In some examples, the roller 3002 is coupled to the second end 106 of the body 102 via friction fit. For example, in the illustrated example, the body 3100 includes a flange 3102 disposed in the third opening 120 of the body 102. In some examples, the flange 3102 of the body 3100 is held in the third opening 120 via friction fit, which enables a user to remove and/or replace the roller 3002. Additionally or alternatively, the body 3100 can be coupled to the body 102 via an adhesive (e.g., glue) and/or a mechanical fastener (e.g., a screw or pin). The body 3100 of the roller 3002 includes a shaft 3104 extending downward from the flange 3102. The roller 3000 includes a ball 3106 in the shaft 3104. A portion of the ball 3106 is exposed from an end 3108 of the shaft 3104. The ball 3106 is rotatable (e.g., can spin or roll) in the shaft 3104, but is prevented from being removed from the end 3106 of the shaft 3104 by a shoulder 3110 at the end 3106 of the shaft 3104. In some examples, the ball 3106 is about 0.39 inches (in) in diameter. In other examples, the ball 3106 can be larger or smaller. The body 3100 of the roller 3002 includes a reservoir 3112 that extends upward from the flange 3102. In this example, the reservoir 3112 is disposed in the internal cavity 700. In the illustrated example, the roller 3200 includes a cap 3114 coupled a portion (e.g., a top) of the reservoir 3112. The reservoir 3112 contains an amount of a liquid, such as an essential oil. The liquid fills the shaft 3104 and coats the surface of the ball 3106. The flange 3102 has an extension 3115 that extends inward and into the upper portion of the shaft 3104 to block the ball 3106 from moving into the reservoir 3112. Therefore, the extension 3115 and the shoulder 3110 maintain the ball 3106 in the shaft 3104. As the ball 3106 is rolled along the skin of a user, the liquid is rolled and/or otherwise applied onto the skin of the user. In some examples, the reservoir 3112 contains about 3.5 milliliters (mL) of liquid. In other examples, the reservoir 3112 can be sized to contain more or less liquid.

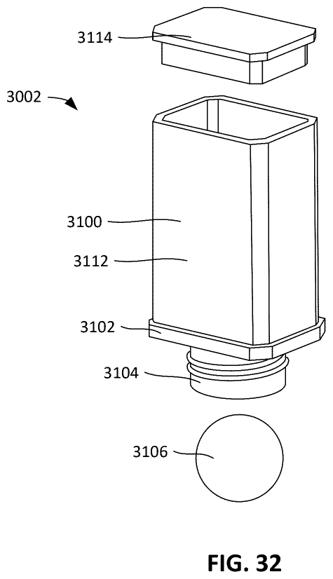

FIG. 32 shows an exploded view of the roller 3002, including the body 3100, the ball 3106, and the cap 3114. In some examples, the body 3100, which includes the flange 3102, the shaft 3104, and the reservoir 3112, is constructed as a single unitary part or component (e.g., an integral monolithic structure). In other examples, the body 3100 can be constructed from multiple parts or components that are coupled together. In some examples, the body 3100 and the cap 3114 are constructed of plastic (e.g., acrylonitrile butadiene styrene (ABS)), and the ball 3106 is constructed of metal (e.g., stainless steel). In some examples, the cap 3114 is removably coupled to the reservoir 3112. For example, the cap 3114 may be held on the top of the reservoir 3112 via friction fit. This enables a user to be able to remove the cap 3114 and re-fill the reservoir 3112. For example, a user can remove (e.g., by pulling) the roller 3002 from the second end 106 of the body 102, and then the user can remove the cap 3114 to re-fill the reservoir 3112 with more of the same or different liquid (e.g., a different essential oil having a different scent). In other examples, the roller 3002 may not be removeable from the body 102. For example, the body 3100 of the roller 3002 can be integrally formed with the body 102 of the inhaler 3000.

Referring back to FIG. 31, the inhaler 3000 can include the first and second pads 708, 710 (or only one pad) that are soaked or saturated in a liquid, such as an essential oil. The nose tips 112, 114 have the openings 116, 118, respectively, that are in fluid communication with the internal cavity 700. The user can insert the nose tips 112, 114 into their nostrils and inhale the fumes or vapors through the openings 116, 118 in the nose tips 112, 114. In this example, the first and second nose tips 112, 114 include the openings 3006a-3006d on the sides of the nose tips 112, 114. Because the third opening 120 is blocked or substantially blocked by the roller 3002, the openings 3006a-3006d allow air flow into the internal cavity 700 when the user is using the inhaler 3000 nasally. This ensures a sufficient amount of air flows through the cavity 700 to carry the vapors or fumes to the user. In some examples, the liquid in the pads 708, 710 can be the same type of liquid as in the roller 3002. For example, the pads 708, 710 may be soaked in a first liquid and the roller 3002 may contain a second liquid, where the first and second liquids may be a same type of a liquid or a different type of liquid. For example, the first and second liquids may be a same type of essential oil having a certain scent or extract. Therefore, the user can nasally inhale the fumes from the essential oil and also apply the essential oil to their skin. In other examples, the first liquid in the pads 708, 710 can be an essential oil having a first scent or extract, and the second liquid in the roller 3002 can be an essential oil having a second scent or extract that is different than the first scent or extract. This allows the user to benefit from different scents or extracts.

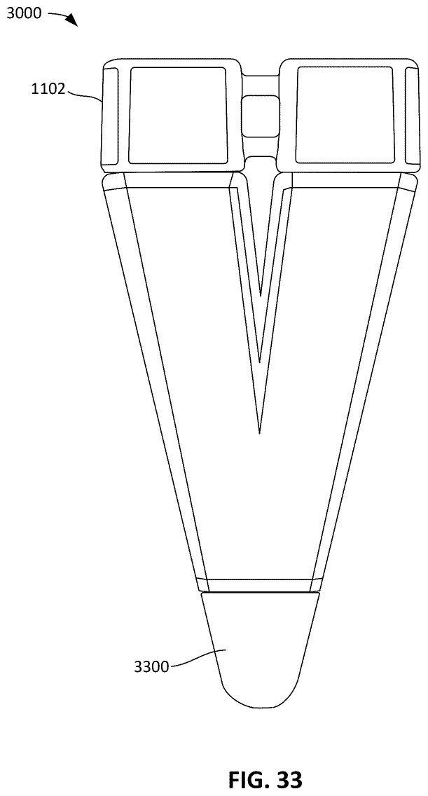

In some examples, the inhaler 3000 can include one or more caps. For example, FIG. 33 shows the inhaler 3000 with the first cap 1102, which is the same as the first cap 1102 from FIG. 11C, and a second cap 3300. The first cap 1102 is disposed on and covers the nose tips 112, 114 (FIG. 30). The second cap 3300 is disposed on and covers the roller 3002 (FIG. 30).

FIG. 34 is a cross-sectional view of the first cap 1102. The first cap 1102 has bores 3400a, 3400b for receiving the first and second nose tips 112, 114. Further, the inner surfaces of the bores 3400a, 3400b have grooves 3402a, 3402b, respectively. As disclosed above, the first and second nose tips 112, 114 can have rings 1106a, 1106b (FIG. 11B, FIG. 30). When the first cap 1102 is placed onto the nose tips 112, 114, the nose tips 112, 114 are inserted into the bores 3400a, 3400b, respectively, and the rings 1106a, 1106b fit into the grooves 3402a, 3402b, respectively, which help to hold the first cap 1102 on the nose tips 112, 114. The first cap 1102 can be removed by pulling on the first cap 1102.

FIG. 35A is a cross-sectional view of the second cap 1104 from FIG. 11C that can be used with the mouthpiece 1100. The second cap 1104 has a bore 3500. The top end of the second cap 1104 has a groove 3502. When the second cap 1104 is placed over the mouthpiece 1100, the mouthpiece 1100 is inserted into the bore 3500, and the ring 1108 (FIG. 11B) fits into the groove 3502, which helps hold the second cap 1104 on the mouthpiece 1100.

FIG. 35B is a cross-sectional view of the second cap 3300 that can be used with the roller 3002. In some examples, the second cap 3300 can be threadably coupled to (e.g., screwed onto) the roller 3002. For example, as shown in FIG. 30, the shaft 3104 of the roller 3002 has the threads 3004. The second cap 3300 has a bore 3504 with internal threads 3506. Therefore, the second cap 3300 can be threadably coupled (e.g., screwed onto) to the roller 3002. In other examples, the second cap 3300 can be coupled to the roller 3002 via other means.

The inhaler 100 (FIG. 1) and the inhaler 3000 (FIG. 30) have the same body 102. As such, a manufacturer can use the same body 102 to produce the inhaler 100, which has a mouth opening 120 or mouthpiece 1100 for oral use, or the inhaler 3000 with the roller 3002 for applying liquid to a surface.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc., may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, or (7) A with B and with C. As used herein in the context of describing structures, components, items, objects and/or things, the phrase “at least one of A and B” is intended to refer to implementations including any of (1) at least one A, (2) at least one B, or (3) at least one A and at least one B. Similarly, as used herein in the context of describing structures, components, items, objects and/or things, the phrase “at least one of A or B” is intended to refer to implementations including any of (1) at least one A, (2) at least one B, or (3) at least one A and at least one B. As used herein in the context of describing the performance or execution of processes, instructions, actions, activities, etc., the phrase “at least one of A and B” is intended to refer to implementations including any of (1) at least one A, (2) at least one B, or (3) at least one A and at least one B. Similarly, as used herein in the context of describing the performance or execution of processes, instructions, actions, activities, etc., the phrase “at least one of A or B” is intended to refer to implementations including any of (1) at least one A, (2) at least one B, or (3) at least one A and at least one B.

As used herein, singular references (e.g., “a”, “an”, “first”, “second”, etc.) do not exclude a plurality. The term “a” or “an” object, as used herein, refers to one or more of that object. The terms “a” (or “an”), “one or more”, and “at least one” are used interchangeably herein. Furthermore, although individually listed, a plurality of means, elements, or actions may be implemented by, e.g., the same entity or object. Additionally, although individual features may be included in different examples or claims, these may possibly be combined, and the inclusion in different examples or claims does not imply that a combination of features is not feasible and/or advantageous.

As used in this patent, stating that any part (e.g., a layer, film, area, region, or plate) is in any way on (e.g., positioned on, located on, disposed on, or formed on, etc.) another part, indicates that the referenced part is either in contact with the other part, or that the referenced part is above the other part with one or more intermediate part(s) located therebetween.

As used herein, connection references (e.g., attached, coupled, connected, and joined) may include intermediate members between the elements referenced by the connection reference and/or relative movement between those elements unless otherwise indicated. As such, connection references do not necessarily infer that two elements are directly connected and/or in fixed relation to each other. As used herein, stating that any part is in “contact” with another part is defined to mean that there is no intermediate part between the two parts.

Unless specifically stated otherwise, descriptors such as “first,” “second,” “third,” etc., are used herein without imputing or otherwise indicating any meaning of priority, physical order, arrangement in a list, and/or ordering in any way, but are merely used as labels and/or arbitrary names to distinguish elements for ease of understanding the disclosed examples. In some examples, the descriptor “first” may be used to refer to an element in the detailed description, while the same element may be referred to in a claim with a different descriptor such as “second” or “third. ” In such instances, it should be understood that such descriptors are used merely for identifying those elements distinctly within the context of the discussion (e.g., within a claim) in which the elements might, for example, otherwise share a same name.

As used herein, “approximately” and “about” modify their subjects/values to recognize the potential presence of variations that occur in real world applications. For example, “approximately” and “about” may modify dimensions that may not be exact due to manufacturing tolerances and/or other real world imperfections as will be understood by persons of ordinary skill in the art. For example, “approximately” and “about” may indicate such dimensions may be within a tolerance range of +/−10% unless otherwise specified herein.

From the foregoing, it will be appreciated that example systems, apparatus, articles of manufacture, and methods have been disclosed that enable an inhaler to be used nasally or orally. This enables the substance, such as an essential oil, to be consumed (e.g., inhaled) through the nose or the mouth, which gives the user the option on how to experience the essential oil and its beneficial effects. The example inhales disclosed herein are small and portable.

Examples and combinations of examples disclosed herein include the following:

Example 1 is an inhaler comprising, a body including a first end, a second end opposite the first end, first and second nose tips on the first end, the first and second nose tips having first and second openings, respectively, a third opening in the second end, and an internal cavity extending between the first and second openings and the third opening. The inhaler also comprises a substance in the internal cavity. The first and second nose tips are to be used to inhale vapors from the substance nasally, and the third opening is to be used to inhale vapors from the substance orally.

Example 2 includes the inhaler of Example 1, further including a valve in the internal cavity, wherein the valve is to block airflow through the internal cavity when suction is applied at the first and second nose tips, and wherein the valve is to open to allow airflow through the internal cavity when suction is applied at the third opening.

Example 3 includes the inhaler of Example 2, wherein the valve is a one-way check valve.

Example 4 includes the inhaler of Example 2, further including a button that is operable to open or close the valve.

Example 5 includes the inhaler of any of Examples 2-4, wherein the valve is disposed between the substance and the third opening.

Example 6 includes the inhaler of any of Examples 2-4, wherein the body is V-shaped.

Example 7 includes the inhaler of Example 6, wherein the body has a first portion and a second portion that are merged at the second end and angled away from each other toward the first end.

Example 8 includes the inhaler of Example 7, wherein the internal cavity includes a first channel that extends through the first portion and a second channel that extends through the second portion.

Example 9 includes the inhaler of Example 8, wherein the substance is a liquid, wherein the inhaler includes a first pad in the first channel and a second pad in the second channel, and wherein the first and second pads are soaked in the liquid.

Example 10 includes the inhaler of any of Examples 7-9, wherein the first and second portions are polyhedrons.

Example 11 includes the inhaler of any of Examples 7-9, wherein the first and second portions are conical.

Example 12 includes the inhaler of any of Examples 1-11, wherein the substance is an essential oil.

Example 13 includes the inhaler of any of Examples 1-12, further including an absorbent material in the internal cavity, wherein the substance is a liquid that is soaked in the absorbent material.

Example 14 includes the inhaler of Example 13, wherein the absorbent material is constructed of a plant-based fiber.

Example 15 includes the inhaler of any of Examples 1-14, further including a mouthpiece coupled to the second end of the body.

Example 16 includes the inhaler of any of Examples 1-15, wherein the first and second nose tips are cylindrical.

Example 17 includes the inhaler of any of Examples 1-16, wherein the inhaler does not include a battery.

Example 18 is an inhaler comprising a body including a first end, a second end, and an internal cavity fluidly coupled to a first opening in the first end and a second opening in the second end. The inhaler comprises a substance in the internal cavity, wherein the first opening is to be used to inhale vapors from the substance nasally, and wherein the second opening is to be used to inhale vapors from the substance orally. The inhaler comprises a valve in the internal cavity, the valve operable between an open state to allow airflow through the internal cavity between the first and second openings, and a closed state to block airflow through the internal cavity between the first and second openings.

Example 19 includes the inhaler of Example 18, wherein the valve is disposed between the substance and the second end.

Example 20 includes the inhaler of Examples 18 or 19, wherein the valve is a one-way check valve.

Example 21 is an inhaler comprising a body including a first end, a second end opposite the first end, an internal cavity, and a nose tip on the first end, the nose tip having an opening in fluid communication with the internal cavity. The nose tip is to be used to inhale vapors from a substance in the internal cavity. The inhaler also includes a roller on the second end. The roller is to be used to dispense a liquid.

Example 22 includes the inhaler of Example 21, wherein the roller includes a shaft and a ball in the shaft, wherein the ball is rotatable in the shaft.

Example 23 includes the inhaler of Examples 21 or 22, wherein the roller includes a reservoir in the internal cavity, the reservoir to contain the liquid.

Example 24 includes the inhaler of Example 23, wherein the roller includes a cap removably coupled to a portion of the reservoir.

Example 25 includes the inhaler of any of Examples 21-24, wherein the liquid is a first liquid and the inhaler includes a polyester rod in the internal cavity, the substance including a second liquid soaked in the polyester rod.

Example 26 includes the inhaler of Example 25, wherein the first and second liquids are a same type of liquid.

Example 27 includes the inhaler of Example 25, wherein the first liquid is a first type of liquid and the second liquid is a second type of liquid different than the first type of liquid.

Example 28 includes the inhaler of any of Examples 21-27, wherein the nose tip has an air flow opening to enable air flow into the internal cavity.

Example 29 includes the inhaler of any of Examples 21-28, further including a cap to be removably coupled to the roller.

Example 30 includes the inhaler of Example 29, wherein the roller includes a shaft with threads, and wherein the cap has a bore with threads, such that the cap can be threadably coupled to the roller.

Example 31 includes the inhaler of any of Examples 21-30, wherein the roller is removably coupled to the body.

Example 32 includes the inhaler of Example 31, wherein the roller is removably coupled to the body via friction fit.

Example 33 includes the inhaler of any of Examples 21-32, further including a cap to be removably coupled to the nose tip.

Example 34 includes the inhaler of Example 33, wherein the nose tip has a ring, and wherein an inner surface of the cap has a groove to receive the ring.

Example 35 is an inhaler comprising a body including a first end, a second end opposite the first end, an internal cavity, and a nose tip on the first end. The nose tip has a first opening in fluid communication with the internal cavity. The nose tip is to be used to nasally inhale vapors from a substance in the internal cavity. The body also includes a second opening in the second end. The second opening is in fluid communication with the internal cavity.

Example 36 includes the inhaler of Example 35, wherein the second opening is to be used to orally inhale vapors from the substance.

Example 37 includes the inhaler of Examples 35 or 36, further including a roller to be coupled to the second end of the body.

Example 38 includes the inhaler of Example 37, wherein, when the roller is coupled to the second end of the body, the roller is disposed in the second opening and extends into the internal cavity.

Example 39 includes the inhaler of Example 38, further including a cap to be removably coupled to the roller.

Example 40 includes the inhaler of Example 39, wherein the roller has a shaft with external threads, and the cap has a bore with internal threads to enable the cap to be threadably coupled to the roller.

The following claims are hereby incorporated into this Detailed Description by this reference. Although certain example systems, apparatus, articles of manufacture, and methods have been disclosed herein, the scope of coverage of this patent is not limited thereto. On the contrary, this patent covers all systems, apparatus, articles of manufacture, and methods fairly falling within the scope of the claims of this patent.

1-20. (canceled)

21. An inhaler comprising:
a body including:
a first end;
a second end opposite the first end;
an internal cavity; and
a nose tip on the first end, the nose tip having an opening in fluid communication with the internal cavity, the nose tip to be used to inhale vapors from a substance in the internal cavity; and
a roller on the second end, the roller to be used to dispense a liquid.

22. The inhaler of claim 21, wherein the roller includes a shaft and a ball in the shaft, wherein the ball is rotatable in the shaft.

23. The inhaler of claim 21, wherein the roller includes a reservoir in the internal cavity, the reservoir to contain the liquid.

24. The inhaler of claim 23, wherein the roller includes a cap removably coupled to a portion of the reservoir.

25. The inhaler of claim 21, wherein the liquid is a first liquid and the inhaler includes a polyester rod, the substance including a second liquid soaked in the polyester rod.

26. The inhaler of claim 25, wherein the first and second liquids are a same type of liquid.

27. The inhaler of claim 25, wherein the first liquid is a first type of liquid and the second liquid is a second type of liquid different than the first type of liquid.

28. The inhaler of claim 21, wherein the nose tip has an air flow opening to enable air flow into the internal cavity.

29. The inhaler of claim 21, further including a cap to be removably coupled to the roller.

30. The inhaler of claim 29, wherein the roller includes a shaft with threads, and wherein the cap has a bore with threads, such that the cap can be threadably coupled to the roller.

31. The inhaler of claim 21, wherein the roller is removably coupled to the body.

32. The inhaler of claim 31, wherein the roller is removably coupled to the body via friction fit.

33. The inhaler of claim 21, further including a cap to be removably coupled to the nose tip.

34. The inhaler of claim 33, wherein the nose tip has a ring, and wherein an inner surface of the cap has a groove to receive the ring.

35. An inhaler comprising:
a body including:
a first end;
a second end opposite the first end;
an internal cavity;
a nose tip on the first end, the nose tip having a first opening in fluid communication with the internal cavity, the nose tip to be used to nasally inhale vapors from a substance in the internal cavity; and
a second opening in the second end, the second opening in fluid communication with the internal cavity.

36. The inhaler of claim 35, wherein the second opening is to be used to orally inhale vapors from the substance.

37. The inhaler of claim 35, further including a roller to be coupled to the second end of the body.

38. The inhaler of claim 37, wherein, when the roller is coupled to the second end of the body, the roller is disposed in the second opening and extends into the internal cavity.

39. The inhaler of claim 38, further including a cap to be removably coupled to the roller.

40. The inhaler of claim 39, wherein the roller has a shaft with external threads, and the cap has a bore with internal threads to enable the cap to be threadably coupled to the roller.
